(19) United States
(12) Patent Application Publication
(10) Pub. No.: US 2024/0131047 A1
(43) Pub. Date: Apr. 25, 2024

(54) SMALL MOLECULE INHIBITORS OF CD38 AS IMMUNOSUPPRESSANTS

(71) Applicant: OHIO STATE INNOVATION FOUNDATION, Columbus, OH (US)

(72) Inventors: Sylvester Michael BLACK, Columbus, OH (US); Bryan Alan WHITSON, Columbus, OH (US); Blake Robert PETERSON, Columbus, OH (US); Austin SCHENK, Columbus, OH (US)

(21) Appl. No.: 18/276,397

(22) PCT Filed: Feb. 8, 2022
(86) PCT No.: PCT/US2022/015670

§ 371 Date: Aug. 8, 2023

Related U.S. Application Data
(60) Provisional application No. 63/147,234, filed on Feb. 8, 2021.

(51) Int. Cl. A61K 31/7076; A61K 31/192; A61K 31/352; A61K 31/4709; A61K 31/496; A61K 31/7048; A61P 29/00; A61P 31/06

(52) U.S. Cl. CPC A61K 31/7076; A61K 31/192; A61K 31/352; A61K 31/4709; A61K 31/496; A61K 31/7048; A61P 29/00; A61P 31/06

(57) ABSTRACT

In one aspect disclosed herein are small molecule inhibitors of CD38 and their use in treating, inhibiting, decreasing, reducing, ameliorating and/or preventing graft versus host disease, ischemia reperfusion injury, graft rejection, inflammatory injury to a graft or a graft recipient. Additionally, disclosed herein are methods of inhibiting CD38 pathway and activity comprising contacting a cell expressing CD38 or administering to a subject in need of CD38 inhibition, any of the small molecule CD38 inhibitors disclosed herein.

### CROSS-REFERENCE TO RELATED APPLICATIONS

This application claims the benefit of priority to U.S. Provisional Application No. 63/147,234, filed Feb. 8, 2021, which is hereby incorporated herein by reference in its entirety.

### STATEMENT REGARDING FEDERALLY SPONSORED RESEARCH OR DEVELOPMENT

This disclosure was made with Government Support under Grant No. R01CA211720 awarded by National Cancer Institute. The Government has certain rights to this disclosure.

### BACKGROUND

Transplantation is the only effective treatment for end-stage organ failure, and access to this procedure for patients continues to be limited by the availability of suitable donor organs. There have been many attempts to increase the availability of donor organs for transplantation, including but not limited to living donor transplantation, donation after cardiac death (PCD), split donor organs (liver), machine perfusion, and the aggressive use of extended criteria and marginal donor organs. Despite these measures, in many areas of the country, there is significant waitlist mortality. Furthermore, secondary to the limited availability of suitable donor organs, organ transplantation is used in only a small fraction of potential patients, which results in rationing of this already scarce resource. Thus, the true impact of transplantation on overall mortality associated with end-stage organ failure secondary to limited donor organ availability is greatly underestimated. Compounding the problem of limited donor organ access, donor organ quality is important, especially when considering extended criteria and marginal donor organs, which have enhanced susceptibility to ischemia-reperfusion injury (IRI) and subsequent graft dysfunction. Determining donor organ “suitability” for transplantation, limiting organ injury, and rescuing those organs that are not considered usable today is critically important for increasing access to transplantation. What are needed are new therapeutic targets, treatments, and treatment methodologies that can address these issues.

### SUMMARY

Provided herein are small molecule inhibitors of CD38 and methods of their use.

In one aspect, disclosed herein are methods of treating, inhibiting, decreasing, reducing, ameliorating and/or preventing the donor organ or tissue (such as, for example, heart, liver, lung, pancreas, kidney, skin, trachea, bone marrow, tendons, cornea, vascular tissue, heart valves, and/or intestines) rejection in a subject in need thereof, the method comprising administering an effective amount of a small molecule CD38 inhibitor to improve the organ or tissue transplant outcome.

Also disclosed herein are methods of preparing a donor organ or tissue (such as, for example, heart, liver, lung, pancreas, kidney, skin, trachea, bone marrow, tendons, cornea, vascular tissue, heart valves, and/or intestines) for transplant comprising administering to the subject an effective amount of a small molecule CD38 inhibitor.

In one aspect, disclosed herein are methods of treating, inhibiting, decreasing, reducing, ameliorating and/or preventing inflammatory injury to a donor organ or tissue (such as, for example, heart, liver, lung, pancreas, kidney, skin, trachea, bone marrow, tendons, cornea, vascular tissue, heart valves, and/or intestines) in a recipient subject comprising administering to the subject an effective amount of a small molecule CD38 inhibitor.

Also disclosed herein are the methods of treating, inhibiting, decreasing, reducing, ameliorating and/or preventing the donor organ or tissue rejection of any preceding aspect, the methods of preparing a donor organ or tissue for transplant of any preceding aspect, and/or the methods of treating, inhibiting, decreasing, reducing, ameliorating and/or preventing inflammatory injury to a donor organ or tissue rejection of any preceding aspect, wherein the subject is resistant or has acquired resistance to immunosuppressive treatment with one or more of eculizumab, thymoglobulin, bortezomib, carfilzomib, basiliximab, mycophenolate mofetil, tacrolimus, or corticosteroids.

In one aspect, disclosed herein are the methods of treating, inhibiting, decreasing, reducing, ameliorating and/or preventing the donor organ or tissue rejection of any preceding aspect, the methods of preparing a donor organ or tissue for transplant of any preceding aspect, and/or the methods of treating, inhibiting, decreasing, reducing, ameliorating and/or preventing inflammatory injury to a donor organ or tissue rejection of any preceding aspect, wherein the small molecule CD38 inhibitor is administered before tissue or organ transplantation, at the time of tissue or organ transplantation, or after tissue or organ transplantation. In some aspects, the CD38 small molecule inhibitor is administered to the donor organ or tissue ex vivo. In some aspects, the CD38 small molecule inhibitor is administered to the subject receiving the donor organ or tissue.

Also disclosed herein are methods of treating, inhibiting, decreasing, reducing, ameliorating and/or preventing graft versus host disease (GvHD) in a subject comprising administering to the subject an effective amount of a small molecule CD38 inhibitor.

In one aspect, disclosed herein are methods of treating, inhibiting, decreasing, reducing, ameliorating and/or preventing ischemia reperfusion injury (IRI) in a subject comprising administering to the subject an effective amount of a small molecule CD38 inhibitor.

Also disclosed herein are methods of treating, inhibiting, decreasing, reducing, ameliorating and/or preventing CD38 activity of a cell expressing CD38 comprising contacting the cell with an effective amount of a small molecule CD38 inhibitor. In some aspects, the cell is in a subject. Thus, for example, disclosed herein disclosed herein are methods of treating, inhibiting, decreasing, reducing, ameliorating and/or preventing CD38 activity in a subject comprising administering to the subject an effective amount of a small molecule CD38 inhibitor.

In one aspect, disclosed herein are methods of treating an inflammatory disease comprising administering to a subject with an inflammatory disease an effective amount of a small molecule CD38 inhibitor.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows an image stream of CD38 localization on primary rat hepatocytes

FIGS. 2A-2E show (2A) a 70% liver hilar clamp; (2B) serum ALT; (2C) H&E of 78C treated mouse after 1 hr IRI and 6 hr reperfusion. (2D-2E) show inhibition of CD38 prevents (2D) TNF-α and (2E) IL-1β release in a mouse model of IRI. 70% liver hilar clamp model with 60 minutes of ischemia followed by 6 hours of reperfusion demonstrated significantly less IL-1β and TNF-α in the plasma when compared with controls, indicating less inflammation in 78C treated animals.

FIG. 3 shows a schematic illustration of nanoprecipitation process for preparation of GalNAc-PLEA nanoparticles.

FIGS. 4A-4B show (4A) a transmission electron microscope image showing the particles size of the PEG-PLGA nanoparticles prepared by Bulk Spray. (4B) loading efficiency of PEG-PLGA loaded with CD38 inhibitor (78C) using a Bulk spray (BS) at pH 9.3 and Electrospray (ES) at pH 7. Different drug to polymer ratio at 0, 0.05, 0.1, 0.25 and 0.5 were studied, n=3* All SD≤1%.

FIG. 5 shows the X-ray crystal structure of CD38 (PDB 4xjs) docked to 78C.

FIGS. 6A-6B show (6A) representative images of free fluorescent Cy5 and. Cy5-loaded Naked and GalNAc targeted nanoparticles in the HepG2 cells after 2 h incubation with the Cy5 concentration 10.0 μM. (6B) IVIS images of organ distribution in mice at 4 h after intravenous injection of Cy5, Naked NP/Cy5 and Targeted NP/Cy5.

FIG. 7 shows the effect of primary hepatocyte cell viability and CD38 activity following 3 hr hypoxia and 3 hr reoxygenation.

FIG. 8 shows the viability of mouse kupffer cells (a phagocytic macrophage cell in liver parenchyma) exposed to hypoxia-reoxygenation and treated with increasing dose of 78c.

FIG. 9 shows TNF-a expression in cell culture supernatant of mouse kupffer cells (a phagocytic macrophage cell in liver parenchyma) exposed to hypoxia-reoxygenation and treated with increasing dose of 78c.

FIG. 10 shows representative photographs of skin allografts 17 days post-transplant and heart allografts 7 days post-transplant.

FIG. 11A-11B depicts graphs showing 78c inhibits proliferation of (11A) CD4 and (11B) CD8 T cells in a dose dependent manner. C57BL6/J mouse splenocytes were labeled. with proliferation dye and cultured for 72 hours in RMPI 1640 with 10% FBS, 2 mM L glutamine, 50 U/ml Penicillin-50 ug/ml Streptomycin, 50 uM b-mercaptoethanol and 1% ITS at a concentration of 100,000 cells per 250 uL in 96 well plates pre-coated with αCD3 (2 ug/ml) and αCD28(5ug/ml). At 0, 24 and 48 hours cells were treated with the αCD38 inhibitor 78c (resuspended in DMSO), DMSO alone, or left untreated. After 72 hours flow cytometry was used to quantify cellular proliferation of CD4 and CD8 T cells. Data are representative of an experiment completed 5 times.

FIG. 12 shows inhibition of CD38 prevents TNF release. Macrophages (THP-1 cell line—M1—inflammatory type)—Exposed to hypoxia and re-oxygenation and treated with 78C released significantly less TN compared with controls. This effect was specific to the 78C when compared with other less specific CD38 inhibitors such as Luteolinidin or Kuromanin.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do riot preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless Specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

“Small molecule” as used herein, is meant to refer to a composition, which has a molecular weight of less than 5 kD (e.g., less than 4 kD, less than 3 kD, less than 2 kD, less than 1 kD, less than 900 D, less than 800 D, less than 700 D, less than 600 D, less than 500 D, less than 400 D, less than 300 D, less than 200 D, less than 100 D). Small molecules can be nucleic acids, peptides, polypeptides, peptidomimetics, carbohydrates, lipids or other organic (carbon-containing) or inorganic molecules.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. Ph in Formula I refers to a phenyl group.

The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained. or branched saturated hydrocarbon moieties. Unless otherwise specified, C₁-C₂₄ (e.g., C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-dimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfa-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g.,, an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, trimethyl-2-propenyl, 1-ethyl-1-methyl-2-propenyl, 1-ethyl-2-methyl-1-propenyl, and 1-ethyl-2-methyl-2-propenyl, The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, -propynyl, 2-propynyl (or propargyl), 1-butyryl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-peritynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butyryl, 1,2-dimethyl-3-butynyl, 2,2-dimethyl-3-butyryl, 3,3-dimethyl-1-butynyl, 1-ethyl-2-butyryl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfa-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both, Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide, In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z′—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁C₁₆, C₁-C₁₂, C₁-C₁₀, C₁-C₈, C₁C₆, C₁-C₄) alkyl group are intended. Examples include methoxy, ethoxy, propoxy, 1-methyl-ethoxy, butoxy, 1-methyl-propoxy, 2-methyl-propoxy, 1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2-trimethyl-propoxy, 1-ethyl-1-methyl-propoxy, and 1-ethyl-2-methyl-propoxy.

The term “aldehyde” as used herein is represented by the formula C(O)H.

The terms “amine” or “amino” as used herein are represented by the formula NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above, “Amido” is C(O)NZ¹Z².

The term “carboxylic acid” as used herein is represented by the formula C(O)OH. A “carboxylate” or “carboxyl” group as used herein is represented by the formula C(O)O⁻.

The term “ester” as used herein is represented by the formula —OC(O)Z¹ or C(O)OZ², where Z′ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula OH.

The term “nitro” as used herein is represented by the formula NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula S(O)—₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(—O—)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

### Methods of Treating

Cluster of differentiation 38 (CD38) is a cell surface glycoprotein found on the surface of multiple types of immune cells including lymphocytes of the thymus, B lymphocytes, and natural killer cells, CD38 is a highly conserved, multifunctional ectoenzyme/endoenzyme with essential roles in nucleotide metabolism (NAD, NADP), leukocyte trafficking, intracellular calcium homeostasis, inflammation, reactive oxygen species (ROS) generation, and cell survival. One of its functions is to activate B cells and T cells, making it a regulator of immune responses. CD38 is an enzyme that synthesizes the calcium-releasing second messengers cyclic ADP-ribose (cADPR) and nicotinic acid adenine dinucleotide phosphate (NAADP). CD38 is also a receptor that can bind CD31 on the surface of T cells to activate those cells to produce cytokines. CD38 catalyzes the synthesis of ADP ribose (ADPR) and cyclic ADP-ribose (cADPR) from NAD+, and it is considered a major regulator of NAD+. CD38 also hydrolyzes cADPR to ADPR, and under acidic conditions, when nicotinic acid is present, CD38 can hydrolyze nicotinamide adenine dinucleotide phosphate (NADP+) to NAADP. These reaction products contribute to regulation of intracellular Ca2+. In terms of subcellular localization, CD38 is an ectoezyme on cell outer surfaces, but it is also found on the inner surface of cell membranes, where it faces the cytosol and executes the same enzymatic functions. The loss of CD38 function is associated with impaired immune responses. On natural killer cells, CD38 binds CD31 on endothelial cells for attachment to the endothelium. On leukocytes, CD38 binds CD16 on endothelial cells for leukocyte attachment to blood vessel walls, facilitating the passage of leukocytes through blood vessel walls. In macrophages, expression of CD38 is induced by the cytokine interferon gamma and lipopolysaccharide. In monocytes, interferon gamma also strongly induces expression of CD38. CD38 inhibitors are used as therapeutics for the treatment of asthma. Daratumumab (Darzalex), an antibody that targets CD38, has been used to treat multiple myeloma.

CD38 expression and enzymatic activity are highly increased in IRI, acute, and chronic inflammatory processes. Therefore, given the prominent role of CD38 in inflammation and cellular response to injury, cell-specific inhibition of CD38 can protect donor livers against injury and graft dysfunction during transplantation while limiting off-target effects and toxicity.

The present disclosure provides methods for methods of treating, inhibiting, decreasing, reducing, ameliorating and/or preventing the donor organ or tissue rejection; methods of preparing a donor organ or tissue for transplant; methods of treating, inhibiting, decreasing, reducing, ameliorating and/or preventing inflammatory injury to a donor organ or tissue rejection; methods of treating, inhibiting, decreasing, reducing, ameliorating and/or preventing graft versus host disease (GvHD); methods of treating, inhibiting, decreasing, reducing, ameliorating and/or preventing ischemia reperfusion injury (IRI); and or methods of treating, inhibiting, decreasing, reducing, ameliorating and/or preventing an inflammatory disease in a subject in need thereof. The method including administering to a recipient subject and/or a donor organ or tissue an effective amount of a small molecule CD38 inhibitor.

The method disclosed herein include administering to a subject in need thereof (i.e., a recipient subject) and/or a donor tissue or organ an effective amount of a small molecule CD38 inhibitor to improve organ transplant outcome. Small molecule CD38 inhibitors can be classified as NAD-analogs, flavonoids and heterocycles compounds. Small molecule CD38 inhibitors can be covalent and non-covalent inhibitors. Covalent inhibitors generally form a bond in the active site at Glu226. On the other hand, non-covalent inhibitors generally bind to amino acid resides in the active site of the enzyme through weaker interactions, like hydrogen and hydrophobic bonds.

In some embodiments, the small molecule CD38 inhibitor can be defined by Formula I:

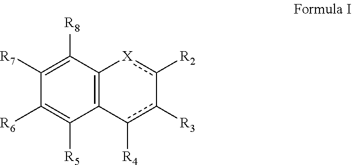

wherein

X is O, or NR₁; R₁ is absent, H, ether, thioether, amine, ester, amide, halogen, or substituted or unsubstituted alkyl, heteroalkyl, alkenyl, alkynyl;

R₂ is a substituted or unsubstituted alkyl, cycloalkyl, heteroalkyl, heterocycloalkyl, heteroaryl, aryl, alkylaryl; ether, thioether, amine, ester, amide, halogen, or oxo;

R₃ is H, substituted or unsubstituted alkyl, alkoxy, cycloalkyl, heterocycloalkyl; or a glycoside,

R₄ is a H, oxo, —NR₁₀R₁₁; wherein R₁₀ and R₁₁ are each independently H, substituted or unsubstituted alkyl, aryl, cycloalkyl, or alkylaryl; and

R₅-R₈ are each independently H, —OH, halogen, amide, substituted or unsubstituted alkyl, substituted or unsubstituted aryl, substituted or unsubstituted cycloalkyl, substituted or unsubstituted alkylaryl, or substituted or unsubstituted heteroaryl.

In some embodiments, R₂, and/or at least one of Rio and RH are each independently defined by the Formula Ia:

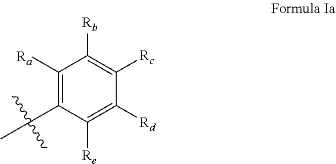

wherein
Rₐ-R_{d} are each independently substituted with H, —OH, —SH, ether, thioether, amine, ester, carboxylic acid, amide, sulfonyl, phosphate, nitro, halogen, nitrile, trifluoromethyl, or substituted or unsubstituted alkyl, heteroalkyl, alkenyl, alkynyl, or alkoxy. In some embodiments, Rₐ-R_{d} are each independently substituted with H, —OH, halogen, trifluoromethyl, substituted or unsubstituted alkyl. In some embodiments, when X is O and R₅ and R₇ are —OH, then R₂, is defined by Formula Ia and R_{b} and R_{c} are —OH. In some embodiments, when X is O and R₅ and R₇ are —OH, then R₂ is defined by Formula Ia and R_{c} is —OH.

In some embodiments, the small molecule CD38 inhibitor can be defined by Formula Ib:

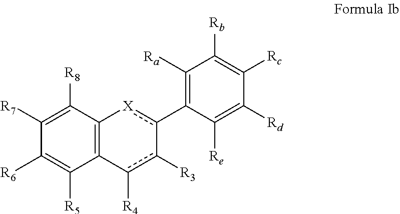

wherein

X is O, or NR₁; R₁ is absent, H, ether, thioether, amine, ester, amide, halogen, or substituted or unsubstituted alkyl, heteroalkyl, alkenyl, alkynyl;

R₃ is H, substituted or unsubstituted alkyl, alkoxy, cycloalkyl, heterocycloalkyl; or a glycoside,

R₄ is a H, oxo, 'NR₁₀R₁₁; wherein R₁₀ and R₁₁ are each independently H, substituted or unsubstituted alkyl, aryl, cycloalkyl, or alkylaryl;

R₅-R₈ are each independently H, —OH, halogen, amide, substituted or unsubstituted alkyl.

Rₐ-R_{d} are each independently substituted with H, —OH, —SH, ether, thioether, amine, ester, carboxylic acid, amide, sulfonyl, phosphate, nitro, halogen, nitrite, trifluoromethyl, or substituted or unsubstituted alkyl, heteroalkyl, alkenyl, alkynyl, or alkoxy. In some embodiments, Rₐ-R_{d} are each independently substituted with H, —OH, halogen, trifluoromethyl, substituted or unsubstituted alkyl. In some embodiments, X is O; R₅ and R₇ are —OH; R₃, R₄, R₆, R₈, Rₐ, R_{d}, and Rₑ are H; and R_{b} and R_{c} are —OH. In some embodiments, X is O; R₅ and R₇ are —OH; R₄ is oxo; R₃, R₆, R₈, Rₐ, R_{b}, R_{d}, and Rₑ are H; and R_{c} is —OH.

In some embodiments, the small molecule CD38 inhibitor can be defined by Formula Ic:

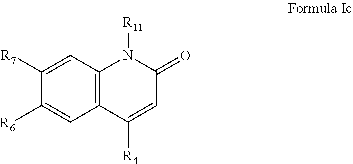

wherein

R₄ is a H, oxo, or —NR₁₀R₁₁;

R₆-R₇ are each independently H, —OH, halogen, amide, substituted or unsubstituted alkyl, substituted or unsubstituted aryl, substituted or unsubstituted cycloalkyl, substituted or unsubstituted alkylaryl, or substituted or unsubstituted heteroaryl; and

R₁₀ and R₁₁ are each independently H, substituted or unsubstituted alkyl, substituted or unsubstituted aryl, substituted or unsubstituted cycloalkyl, substituted or unsubstituted heterocycloalkyl, or substituted or unsubstituted alkylaryl.

In some embodiments, the small molecule CD38 inhibitor can be defined by Formula II:

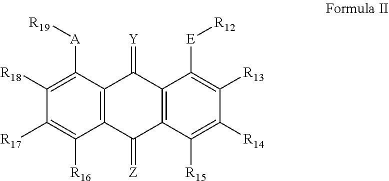

wherein

Y and Z are independently O or S;

A and E are independently O or S;

R₁₂ and R₁₉ are independently substituted or unsubstituted H, alkyl, alkenyl, aryl or alkylaryl;

R₁₃-R₁₈ are independently hydrogen, —OH, —SH, ether, thioether, amine, ester, carboxylic acid, amide, sulfonyl, phosphate, nitro, halogen, mitrile, trifluoromethyl, or substituted or unsubstituted alkyl, heteroalkyl, alkenyl, alkynyl;

wherein substituents for the substituted groups are independently halogen, hydroxyl, thiol, alkyl, alkenyl, alkynyl, aryl, heteroaryl, cycloalkyl, heterocycloalkyl, alkoxy, cyano, carbonyl, amino, amino, sulfonyl, sulfonic acid, phosphoryl, or phosphonyl; and pharmaceutically acceptable salts thereof.

In some embodiments, the small molecule CD38 inhibitor can be defined by Formula III:

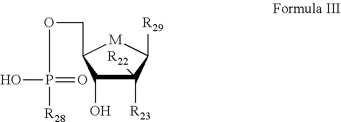

wherein

M is O or C;

R₂₂ and R₂₃ are independently H, —OH, or halogen;

R₂₈ is a —OH, nucleotide derivative, or a substituent as defined by the Formula IIIb:

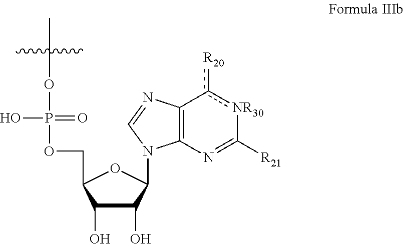

wherein

R₂₀ is O, NH, or NH₂;

R₂₁ is halogen, substituted or unsubstituted alkyl, aryl, cycloalkyl, alkylaryl, or heteroaryl; and

R₃₀ is absent, H, substituted or unsubstituted alkyl, aryl, cycloalkyl, alkylaryl, or heteroaryl; and

R₂₉ is —OH; substituted or unsubstituted alkyl, aryl, cycloalkyl, alkylaryl, or heteroaryl; or an enzyme; or a substituent as defined by the Formula IIIa:

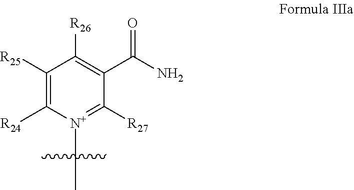

wherein

R₂₄-R₂₇ are independently hydrogen, —OH, —SH, ether, thioether, amine, ester, carboxylic acid; amide, sulfonyl, phosphate, nitro, halogen, mitrile, trifluoromethyl, or substituted or unsubstituted alkyl, heteroalkyl, alkenyl, alkynyl.

In some embodiments, the small molecule CD38 inhibitor can be defined by Formula IV:

wherein

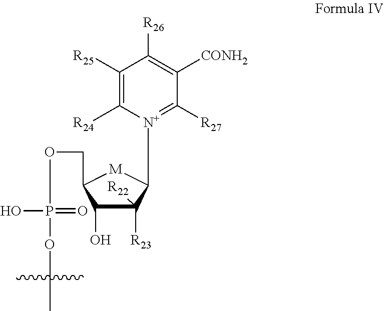

M is O or C;

R₂₂ and R₂₃ are independently H, —OH, or halogen;

R₂₄-R₂₇ are independently hydrogen, —OH, —SH, ether, thioether, amine, ester, carboxylic acid, amide, sulfonyl, phosphate, nitro, halogen, mitrile, trifluoromethyl, or substituted or unsubstituted alkyl, heteroalkyl, alkenyl, alkynyl; and

R₂₈ is a —OH, nucleotide derivative, or a substituent defined by Formula IIIb.

In some embodiments, the small molecule CSD38 inhibitor can be defined by Formula V:

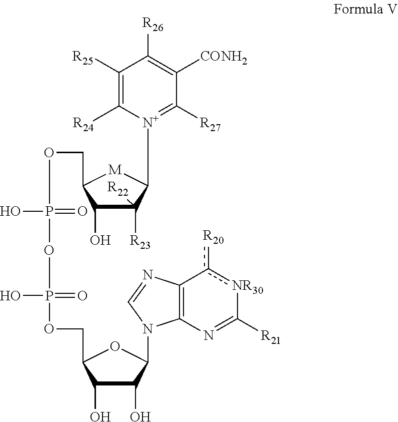

wherein

M is O or C;

R₂₀ is O, NH, or NH₂;

R₂₁ is H, halogen, substituted or unsubstituted alkyl, aryl, cycloalkyl, alkylaryl, or heteroaryl;

R₂₀ and R₂₃ are independently H, —OH, or halogen,

R₂₄-R₂₇ are independently hydrogen, —OH, —SH, ether, thioether, amine, ester, carboxylic acid, amide, sulfonyl, phosphate, nitro, halogen, mitrile, trifluoromethyl, or substituted or unsubstituted alkyl, heteroalkyl, alkenyl, alkynyl; and

R₃₀ is absent, H, substituted or unsubstituted alkyl; aryl, cycloalkyl, alkylaryl, or heteroaryl.

In some embodiments, the small molecule CD38 inhibitor can be defined by Formula VI:

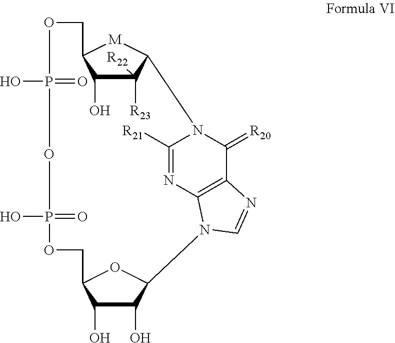

wherein

M is O or C;

R₂₀ is O, NH, or NH₂;

R₂₁ is H, halogen, substituted or unsubstituted alkyl, aryl, cycloalkyl, alkylaryl, or heteroaryl; and

R₂₂ and R₂₃ are independently H, —OH, or halogen.

Examples of suitable small molecule CD38 inhibitors may be found, e.g., in PCT Publication No. WO 2016/087975; PCT Publication No. WO 2013/002879; PCT Publication No. WO 2014/011753; US. Patent Application Publication No. 2020/0046741; Chini, et al., Trends Pharmacol Sci. 39(4): 424-436 (2018); Deshpande D A, et al., Pharmacology & therapeutics, 172, 116-26 (2017); Shrimp J H, et al., J Am Chem Soc.; 136(15):5656-5663 (2014); Sauve A A., J Am Chem Soc., 122(33):7855-59 (2000); Zhang S, et al., Chem Biol and Drug Design, 86(6):1411-24 (2015); Liu Q, et al., Biol Chem., 284(40):27637-27645 (2009); Liu Z, et al., Messenger, 2(1):19-32 (2013); Muller-Steffner H M, et al., J of Biol Chem., 267(14):9606-11 (1992); Slama J T, et al., Biochemistry, 27(1):183-93 (1988); Wall K, et al., The Bioch J., 335:631-636 (1998); Sauve A A, et al., Biochemistry, 41(26):8455-63 (2002); Kwong A K Y, et al., Biochemistry, 51(0:555-64 (2012); Kellenberger E, et al., Bioorg and Med Chem Lett., 21(13):3939-3942 (2011); Escande C, et al., Diabetes, 62(4):1084-93 (2013); Boslett James, et al. J Pharmacol Exp Ther., 361(1):99-108 (2017); Shu B, et al. Cell Signal., 42, 249-258 (2018); Blacher E, et al., Int J Cancer, 136(6):1422-33 (2015); Blacher E, et al., Cancer Cell & Microenv., 2:e486. 1-6 (2015); Schiavoni I, et al., Immunology, 154:1, 122-131 (2018); and Haffner C D, et al. J Med Chem., 58(8):3548-71 (2015), each of which is herein incorporated by reference in its entirety.

In some embodiments, the small molecule CD38 inhibitor can be compounds selected from the following compounds:

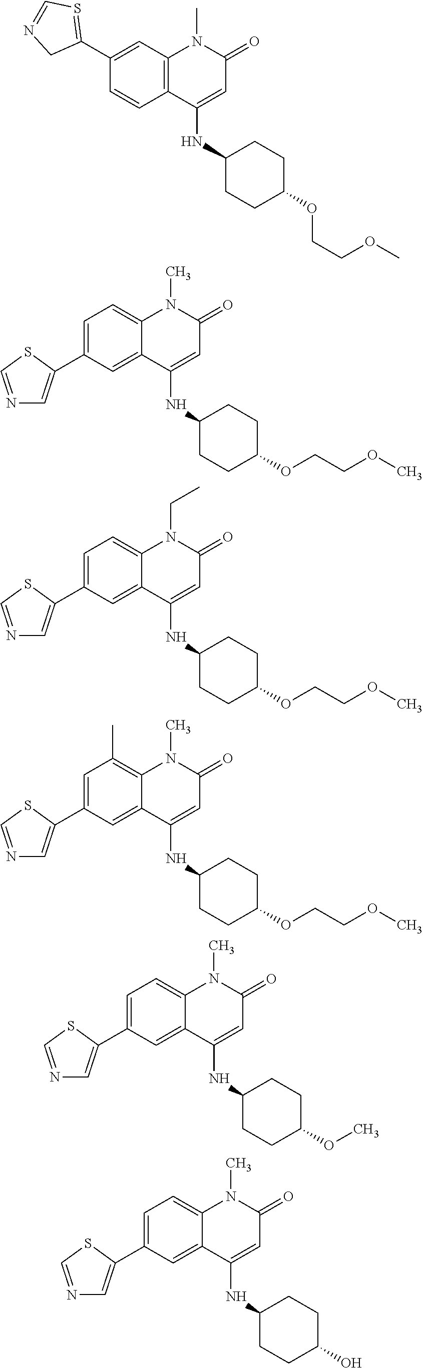

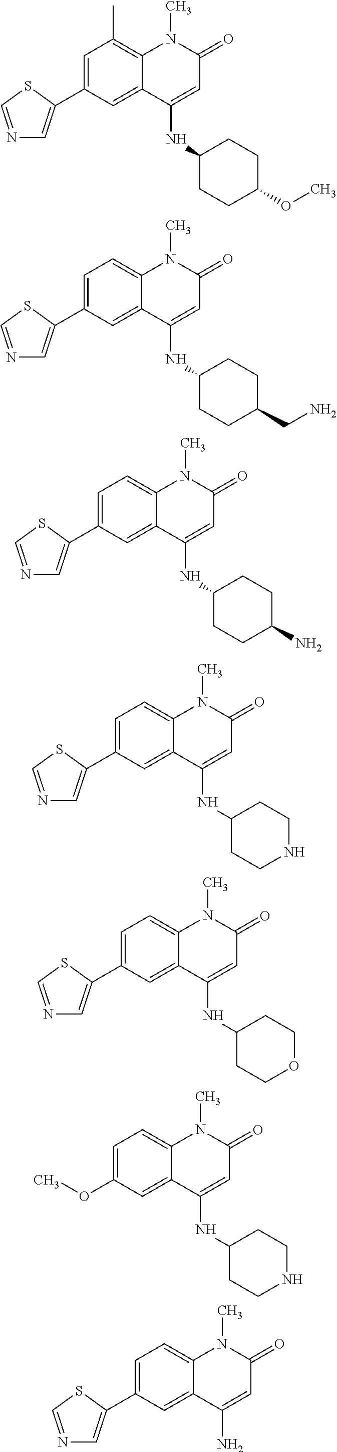

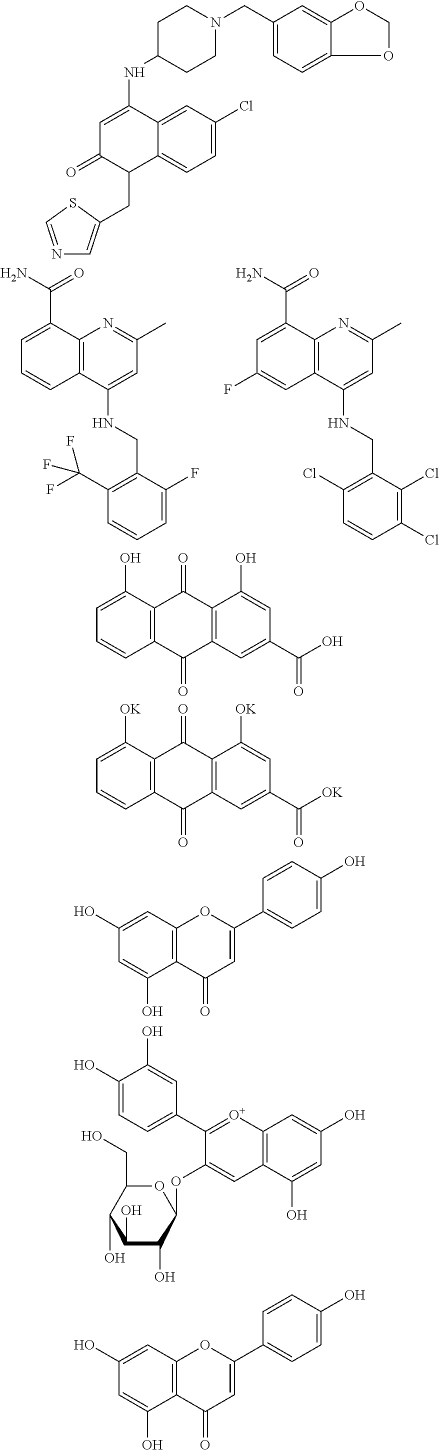

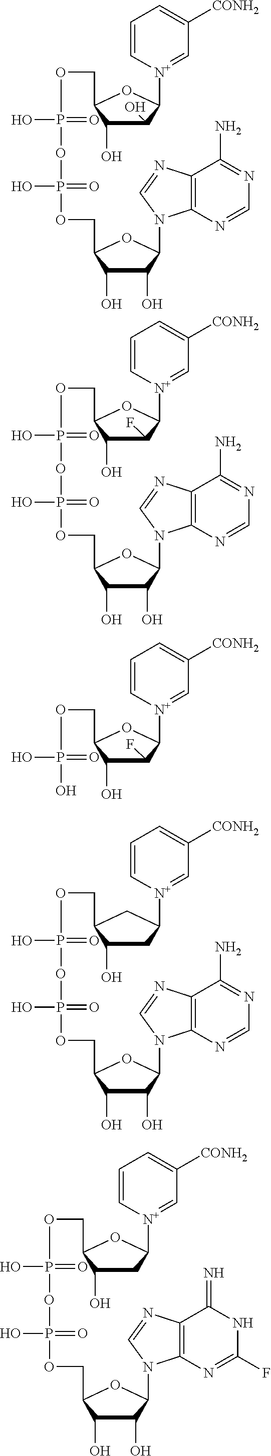

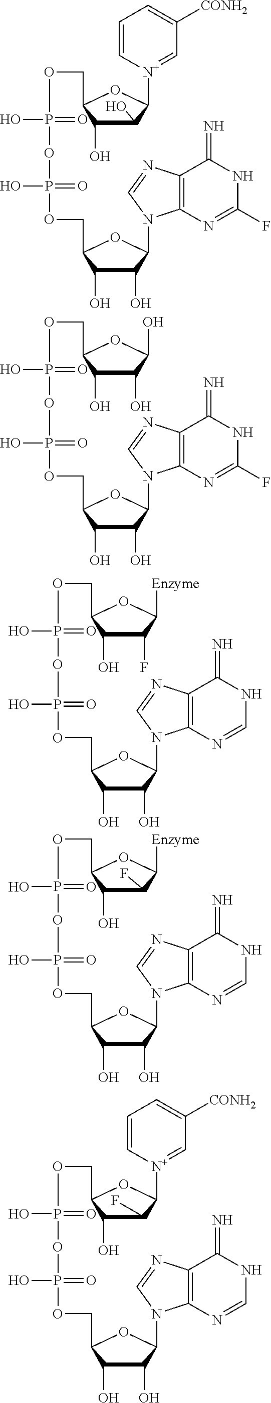

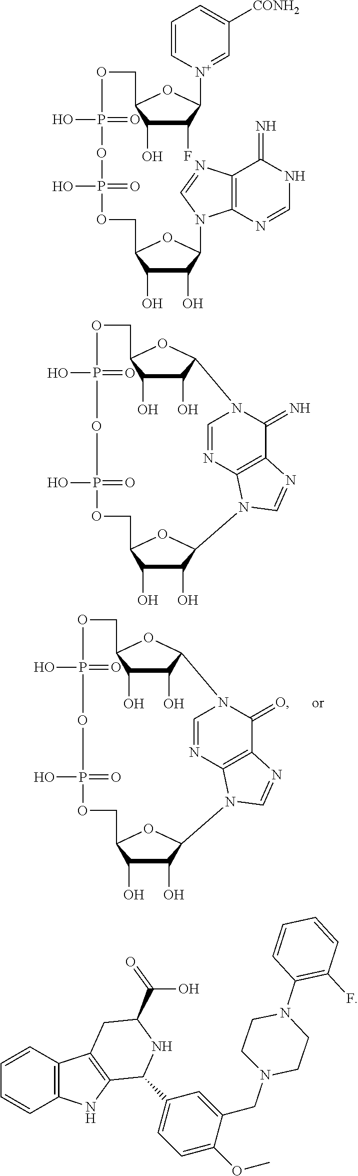

In some embodiments, the small molecule CD38 inhibitor can have a molecular weight of at least 50 D, at least 100 D, at least 200 D, at least 300 D, at least 400 D, at least 500 D, at least 600 D, at least 700 D, at least 800 D, at least 900 D, at least 1 kDa, at least 2 kDa, at least 3 kD, at least 4 kD). In some embodiments, the small molecule CD38 inhibitor can have a molecular weight of 5 kD or less, (e.g., 4 kd or less, 3 kD or less, 2 kD or less, 1 kD or less, 900 D or less, 800 D or less, 700 D or less, 600 D or less, 500 D or less, 400 D or less, 300 D or less, 200 D or less, or 100 D or less).

The small molecule CD38 inhibitor can have a molecular weight ranging from any of the minimum values described above to any of the maximum values described above. For example, in some embodiments, the small molecule CD38 inhibitor can have a molecular weight of from 50 D to 5 kDa, (e.g., from 50 D to 4 kDa, from 50 D to 3 kDa, from 50 D to 2 kDa, from 50 D to 1 kDa, from 50 D to 900 Da, from 50 D to 800 Da, from 50 D to 700 Da, from 50 D to 600 Da, from 50 D to 500 Da, from 50 D to 400 Da, from 50 D to 300 Da, from 50 D to 200 Da, from 50 D to 100 Da, from 100 D to 5 kDa, 100 D to 4 kDa, from 100 D to 3 kDa, from 100 D to 2 kDa, from 100 D to 1 kDa, from 100 D to 900 Da, from 100 D to 800 Da, from 100 D to 700 Da, from 100 D to 600 Da, from 100 D to 500 Da, from 100 D to 400 Da, from 100 D to 300 Da, from 100 to 200 Da, from 200 D to 5 kDa, from 200 D to 4 kDa, from 200 D to 3 kDa, from 200 D to 2 kDa, from 200 D to 1 kDa, from 200 D to 900 Da, from 200 D to 800 Da, from 200 D to 700 Da, from 200 D to 600 Da, from 200 D to 500 Da, from 200 D to 400 Da, from 200 D to 300 Da, from 300 D to 5 kDa, 300 D to 4 kDa, from 300 D to 3 kDa, from 300 D to 2 kDa, from 300 D to 1 kDa, from 300 D to 900 Da, from 300 D to 800 Da, from 300 D to 700 Da, from 300 D to 600 Da, from 300 D to 500 Da, from 300 D to 400 Da, from 400 D to 5 kDa, 400 D to 4 kDa, from 400 D to 3 kDa, from 400 D to 2 kDa, from 400 D to 1 kDa, from 400 D to 900 Da, from 400 D to 800 Da, from 400 D to 700 Da, from 400 D to 600 Da, from 400 D to 500 Da, from 500 D to 5 kDa, 500 D to 4 kDa, from 500 D to 3 kDa, from 500 D to 2 kDa, from 500 D to 1 kDa, from 500 D to 900 Da, from 500 D to 800 Da, from 500 D to 700 Da, from 500 D to 600 Da, from 600 D to 5 kDa, 600 D to 4 kDa, from 600 D to 3 kDa, from 600 D to 2 kDa, from 600 D to 1 kDa, from 600 D to 900 Da, from 600 D to 800 Da, from 600 D to 700 Da, from 700 D to 5 kDa, 700 D to 4 kDa, from 700 D to 3 kDa, from 700 D to 2 kDa, from 700 D to 1 kDa, from 700 D to 900 Da, from 700 D to 800 Da, from 800 D to 5 kDa, 800 D to 4 kDa, from 800 D to 3 kDa, from 800 D to 2 kDa, from 800 D to 1 kDa, from 800 D to 900 Da, from 900 D to 5 kDa, 900 D to 4 kDa, from 900 D to 3 kDa, from 900 D to 2 kDa, from 900 D to 1 kDa, from 1 kD to 5 kDa, from 1 kD to 4 kDa, from 1 kD to 3 kDa, from 1 kD to 2 kDa, from 2 kD to 3 kDa, from 2 kD to 4 kD, from 2 kD to 5 kD, from 3 kD to 4 kD, from 3 kD to 5 kD, or from 4 kD to 5 kDa.

Because increased CD38 expression/activity can be associated with enhanced injury during IRI, it is understood and herein contemplated that inhibiting or reducing CD38 expression/activity can inhibit, reduce, decrease, ameliorate, and/or prevent tissue or organ damage during an ischemic, reperfusion, and/or transplantation. Thus, in one aspect, disclosed herein are methods of preparing a donor organ (such as, for example liver, lung, heart, kidney, trachea, or pancreas) or tissue (bones, skin, tendons, cornea, vascular tissue, or heart valves) for transplantation comprising contacting the organ or tissue with any of the small molecule CD38 inhibitors disclosed herein. For example, disclosed herein are methods of preparing a donor organ or tissue for transplantation comprising contacting the organ or tissue with one or more small molecule CD38 inhibitor, In one aspect, the small molecule inhibitor of CD38 can comprise a thiazoloquin(az)olin(on)e compound (such as, for example compound 78C), apigenin, kuromanin, or luteolinidin. In one aspect the donor tissue or organ is contacted with the CD38 inhibitor ex vivo prior to transplantation.

Also disclosed herein are methods of treating, inhibiting, decreasing, reducing, ameliorating and/or preventing graft versus host disease (GvHD) in a subject comprising administering to the subject an effective amount of a small molecule CD38 inhibitor. Alternatively, it is understood and herein contemplated that GvHD can be inhibited, decreased, reduced, and/or prevented by contacting the donor tissue or organ (including perfusion of the donor tissue or organ) with an effective amount of a small molecule CD38 inhibitor ex vivo prior to transplantation to the subject rather than or in addition to administration to the subject an effective amount of a small molecule CD38 inhibitor.

In one aspect, disclosed herein are methods of treating, inhibiting, decreasing, reducing, ameliorating and/or preventing ischemia reperfusion injury (IRI) in a subject comprising administering to the subject an effective amount of a small molecule CD38 inhibitor.

In one aspect, disclosed herein are methods of treating, inhibiting, decreasing, reducing, ameliorating and/or preventing an inflammatory disease comprising administering to a subject with an inflammatory disease an effective amount of a small molecule CD38 inhibitor.

Also disclosed herein are the methods of treating, inhibiting, decreasing, reducing, ameliorating and/or preventing the donor organ or tissue rejection; methods of preparing a donor organ or tissue for transplant; methods of treating, inhibiting, decreasing, reducing, ameliorating and/or preventing inflammatory injury to a donor organ or tissue rejection; methods of treating, inhibiting, decreasing, reducing, ameliorating and/or preventing graft versus host disease (GvHD); methods of treating, inhibiting, decreasing, reducing, ameliorating and/or preventing ischemia reperfusion injury (IRI); and or methods of treating, inhibiting, decreasing, reducing, ameliorating and/or preventing an inflammatory disease; wherein the subject is resistant or has acquired resistance to immunosuppressive treatment with one or more of eculizumab, thymoglobulin, bortezomib, carfilzomib, basiliximab, mycophenolate mofetil, tacrolimus, or corticosteroids.

In one aspect, disclosed herein are the methods of treating, inhibiting, decreasing, reducing, ameliorating and/or preventing the donor organ or tissue rejection, methods of preparing a donor organ or tissue for transplant, and/or the methods of treating, inhibiting, decreasing, reducing, ameliorating and/or preventing inflammatory injury to a donor organ or tissue rejection, wherein the small molecule CD38 inhibitor is administered before tissue or organ transplantation (including, ex vivo perfusion or contact of the donor tissue or organ and/or administration to the recipient subject), at the time of tissue or organ transplantation, or after tissue or organ transplantation. In some aspects, the CD38 small molecule inhibitor is administered to the donor organ or tissue ex vivo. In some aspects, the CD38 small molecule inhibitor is administered to the subject receiving the donor organ or tissue.

In one aspect the small molecule CD38 inhibitors and/or pharmaceutical compositions comprising said CD38 inhibitors disclosed herein can be delivered to a donor subject comprising the donor tissue or organ prior to removal of the organ (such as, for example liver, lung, heart, kidney, trachea, or pancreas) or tissue (bones, skin, tendons, cornea, vascular tissue, or heart valves) or directly to the donor tissue or organ. In some aspect, the small molecule CD38 inhibitors or pharmaceutical composition is delivered to the organ or tissue via ex vivo organ perfusion (EVOP) including, but not limited to normothermic ex-vivo liver perfusion (NEVLP), solution flush, and/or static storage solution such as for example a cold static storage solution or normothermic solution). In some aspects, the small molecule CD38 inhibitors or pharmaceutical composition can be administered prior to transplantation or as part of a post-transplantation procedure. Accordingly, it is understood and herein contemplated that the organ or tissue can be contacted with one or more small molecule CD38 inhibitors ex vivo for any amount of time sufficient to have an efficacious outcome. In one aspect, the organ or tissue can be contacted with an engineered nanoparticle ex vivo for 1, 2, 3, 4, 5 6, 7, 8, 9, 10, 15, 20, 25, 30, 35, 40, 45, 50, 55, 60, 70, 80, 90, 100, 110, 120, 150, 180 minutes, 4, 5, 6, 7, 8, 9, 10, 11, 12, 13, 14, 15, 16, 17, 18, 19, 20, 21, 22, 23, 24, 30, 36, 42, 48, 60, 72 hours, 4, 5, 6, or 7 days. Similarly, in some instances, efficacious outcomes can occur by administering the one or more small molecule CD38 inhibitors directly to a recipient subject (i.e., the subject receiving the donor organ or tissue). Whether applied directly to the donor organ or tissue or administered to the recipient subject, the small molecule CD38 inhibitor can be administered at the time of organ or tissue transplantation or 1, 2, 3, 4,5 6, 7, 8, 9, 10, 15, 20, 25, 30, 35, 40, 45, 50, 55, 60, 70, 80, 90, 100, 110, 120, 150, 180 minutes, 4, 5, 6, 7, 8, 9, 10, 11, 12, 13, 14, 15, 16, 17, 18, 19, 20, 21, 22, 23, 24, 30, 36, 42, 48, 60, 72 hours, 4, 5, 6, or 7 days prior to transplantation.

In some circumstances, a more efficacious result can be achieved by administering the small molecule CD38 inhibitor to the recipient subject after transplantation. Thus, is some aspects, the small molecule CD38 inhibitor can be administered to the subject 1, 2, 3, 4, 5 6, 7, 8, 9, 10, 15, 20, 25, 30, 35, 40, 45, 50, 55, 60, 70, 80, 90, 100, 110, 120, 150, 180 minutes, 4, 5, 6, 7, 8, 9, 10, 11, 12, 13, 14, 15, 16, 17, 18, 19, 20, 21, 22, 23, 24, 30, 36, 42, 48, 60, 72 hours, 4, 5, 6, 7 days after transplantation. It is understood and herein contemplated that a single dose of a CD38 inhibitor alone may not be sufficient to achieve the desired result of treating, inhibiting, decreasing, reducing, ameliorating and/or preventing the donor organ or tissue rejection, inflammatory injury to a donor organ or tissue, graft versus host disease (GvHD), ischemia reperfusion injury (IRI), CD38 activity, and or inflammatory disease. Accordingly, the small molecule CD38 inhibitor can be administered to the subject at least 1, 2, 3, 4, 5, 6, 7, 8, 9, 10, 11, 12, 13, 14, 15, 16, 17, 18, 19, 20, 21, 22, 23, 24, 28, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, or 300 additional time for the life of the recipient subject. It is understood and herein contemplated that administration of the small molecule CD38 inhibitor is not restricted to a specific period relative to the transplantation (i.e., only before, only during, or only after) but can occur before, during, and after transplantation. It is further understood and herein contemplated that subsequent administration of the small molecule CD38 inhibitor can continue for the life of those recipient subject and thus can be administered to the subject at least 1, 2, 3, 4, 5, 6, 7, 8, 9, 10, 11, 12, 13, 14, 15, 16, 17, 18, 19, 20, 21, 22, 23, 24, 28, 26, 27, 28, 29, 30, 31, 45, 58, 59, 60, 61, 62, 90 days, 3, 4, 5, 6, 7, 8, 9, 10, 11, 12, 13, 14, 15, 16, 17, 18, 24, 30, 36, 42, 48, 54, or 60 months following transplantation.

It is understood and herein contemplated that methodologies that work for preparing a donor tissue or organ for transplantation and avoid CD38 mediated injuries that would otherwise leave the tissue or organ marginal or unsuitable for use as a donor organ can be applied to rescue an otherwise marginal or unsuitable organ or tissue from injury and restore said tissue or organ to a suitable state. Thus, in one aspect, disclosed herein are methods of inhibiting, reducing, or repairing tissue damage to a donor organ (such as, for example liver, lung, heart, kidney, trachea, or pancreas) or tissue (bones, skin, tendons, cornea, vascular tissue, or heart valves) during a transplantation procedure comprising contacting the organ or tissue with any of the CD38 small molecule inhibitor disclosed herein. For example, disclosed herein are methods of inhibiting, reducing, decreasing, ameliorating, preventing, and/or repairing tissue damage to a donor organ or tissue during a transplantation procedure comprising contacting the organ or tissue with one or more small molecule CD38 inhibitors. In one aspect, the small molecule inhibitor of CD38 can comprise a thiazoloquin(az)olin(on)e compound (such as, for example compound 78C), apigenin, kuromanin, or luteolinidin.

The disclosed small molecule CD38 inhibitors can also be used to treat autoinflammatory disorders. As used herein “autoinflammatory disorders refer to disorders where the innate immune response attacks host cells. Examples of autoimmune diseases that can be treated by any of the CD38 inhibitor comprising engineered nanovesicle or pharmaceutical compositions disclosed herein include, but are not limited to asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated. Periodic Syndrome (TRAPS), TNERSF11A-associated hereditary fever disease (TRAPS11), Hyperimmunoglobulinemia D with Periodic Fever Syndrome (HMS), Mevalonate Aciduria (MA), Mevalonate Kinase Deficiencies (MKD), Deficiency of Interleukin-1β (IL-1β) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLPR12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

Also disclosed herein are methods of treating, inhibiting, decreasing, reducing, ameliorating and/or preventing CD38 activity of a cell expressing CD38 comprising contacting the cell with an effective amount of a small molecule CD38 inhibitor. In some aspects, the cell is in a subject. Thus, for example, disclosed herein disclosed herein are methods of treating, inhibiting, decreasing, reducing, ameliorating and/or preventing CD38 activity in a subject comprising administering to the subject an effective amount of a small molecule CD38 inhibitor.

### Methods of Administration

The CD38 inhibitors as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose; kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Plutonic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, tri sodium edetate, calcium disodium edetate ; dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, rhea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylceliulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Plutonic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyactylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (.e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the composition as used in the methods described herein may be administered in combination or alternation with one or more additional active agents. Representative examples additional active agents include anti-inflammatory agents (including steroids and non-steroidal anti-inflammatory agents), anti-coagulant agents, antiplatelet agents, and antiseptic agents.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Hepatocyte Specific CD38 Inhibition to Mitigate Ischemia Reperfusion Injury in Liver Allografts

Although CD38 has been studied as a biomarker of inflammation, its central mechanistic role in inflammation and the cellular response to injury makes it an attractive therapeutic target to ameliorate IRI. CD38 also has a prominent role in cellular bioenergetics as the primary consumer of the nucleotides NAD and NADP. Thus, activated CD38 can be considered a negative regulator of NAD-dependent cell survival pathways by controlling NAD availability, as seen with silent information regulator proteins (sirtuins). This is particularly important in marginal donor organs and organs with significant ischemia. NAD and ATP are generally depleted, leaving the donor organ susceptible to increased injury and subsequent organ dysfunction.

Ischemia-reperfusion is a biphasic injury mechanism, featuring prominently in solid organ transplantation, which is especially important during donor organ procurement, preservation, and graft implantation. IRI occurs universally in organ transplantation and is time-delimited, with a return to normal physiology provided that the cellular defenses are adequate for the magnitude of donor organ injury. Therefore, a targeted and reversible therapeutic intervention administered during this time to inhibit CD38 results in significantly reduced allograft injury and dysfunction.

We have developed a novel therapeutic, based on a GalNAc decorated PEGylated PLGA nanoparticle (NP). NP-based therapeutics selectively deliver the therapeutic agents to cells or target organs. Related polylactic acid-co-glycolic acids (PLGAs) are approved by the FDA for clinical use. PLGA-NPs have been extensively used in medical treatment due to their high stability, high loading efficiency, sustained release, cellular uptake, and biodegradability. A large amount of endosomal escape and release from PLGA's results in high efficiency of the encapsulated molecule. The coupling of PLGA with polyethylene glycol (PEG) polymer is one of the most proficient modifications. PLGAs with PEG show an increased half-life in circulation, secondary to the ability to be disguised from recognition by the immune system.

Asialoglycoprotein receptor (ASGPR) is a Ca2+ dependent human C-type lectin transmembrane receptor expressed in high density on the surface of hepatocytes and minimally present elsewhere in the body. Attributes like access from vascular compartment, rapid internalization, and high affinity make it an ideal target for hepatocyte-specific targeting. The main advantage of ASGPR is its affinity towards various ligands as simple as carbohydrates. Given this aspect, among various ligands, N-acetylgalactosamine (GalNAc), an oligosaccharide, has a high affinity towards ASGPRs, and decoration of nanoparticles with GalNAc is of great importance in the selective delivery of therapeutic agents to hepatocytes via ASGPR receptors.

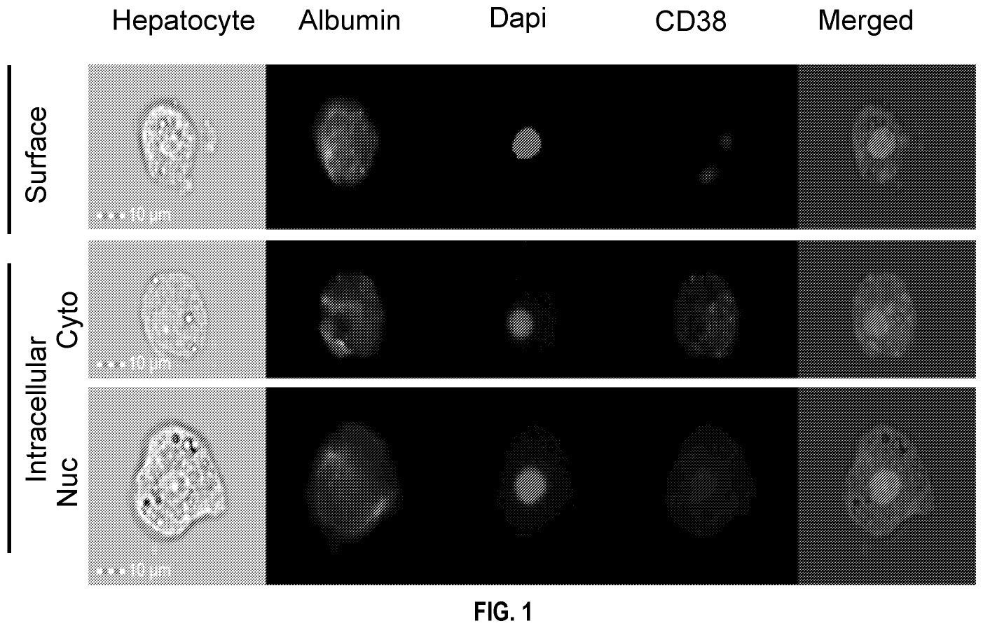

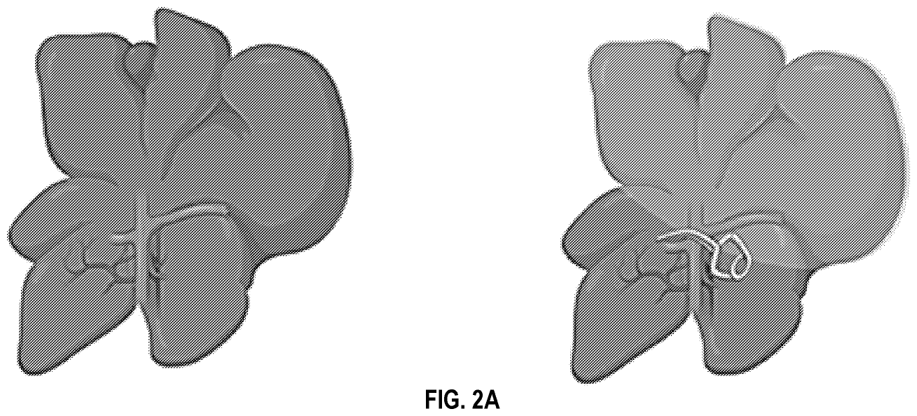

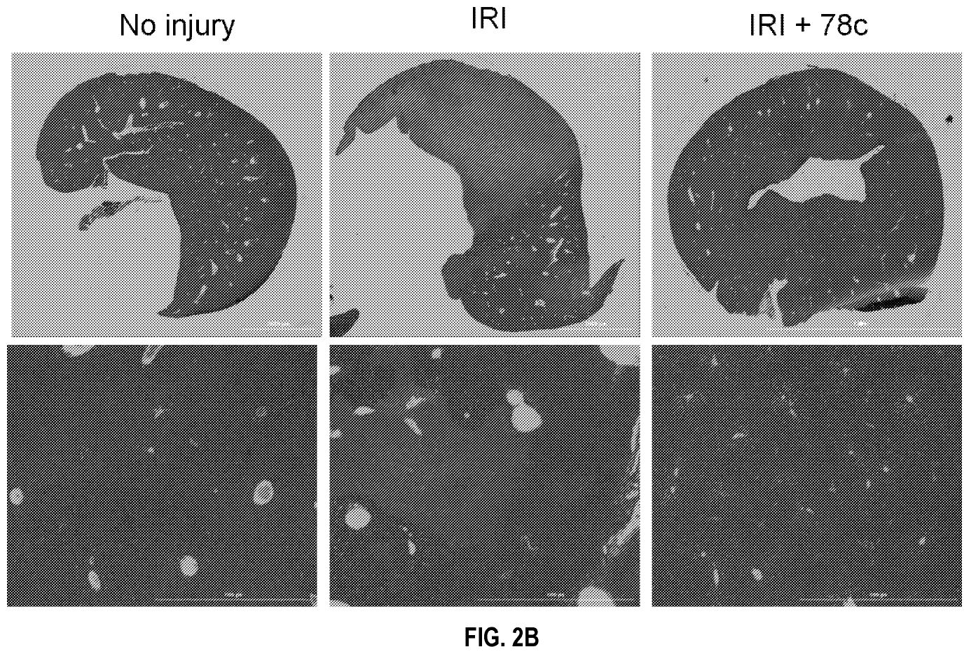

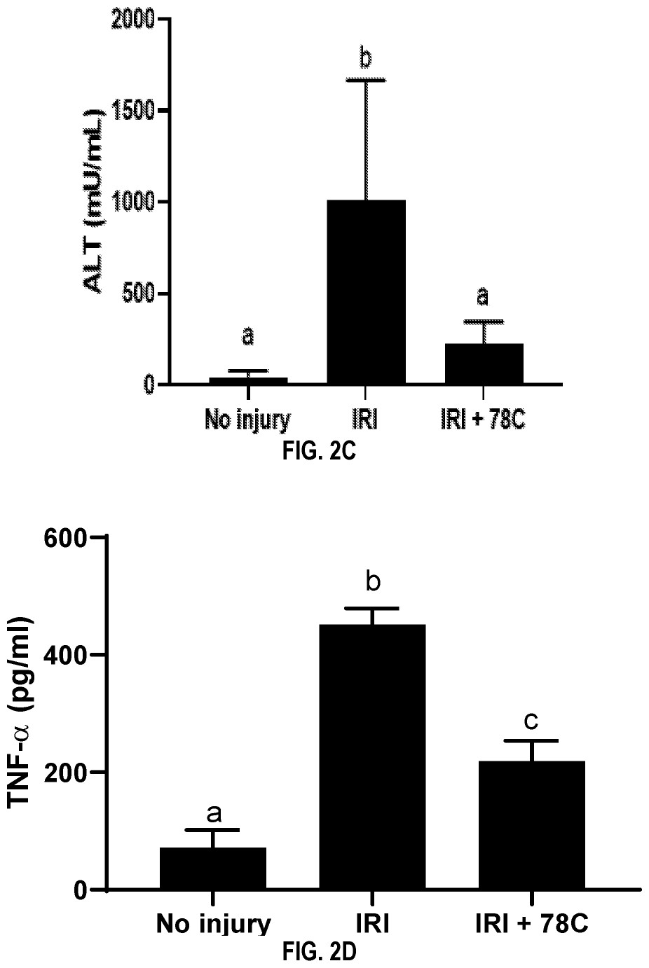

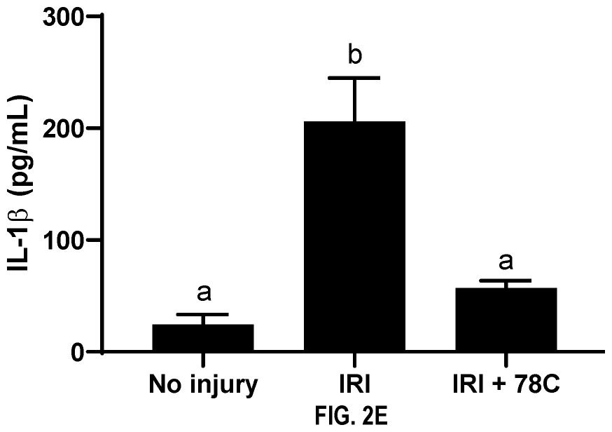

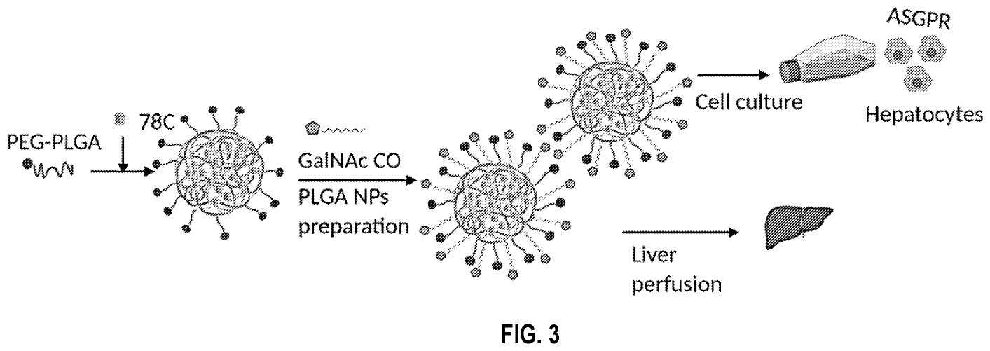

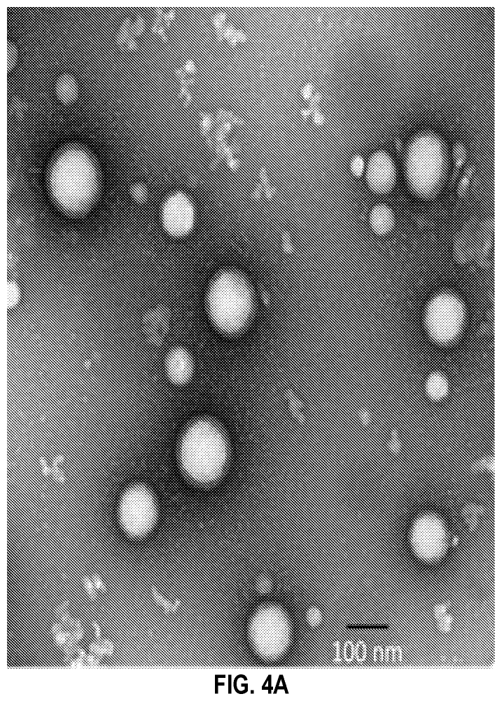

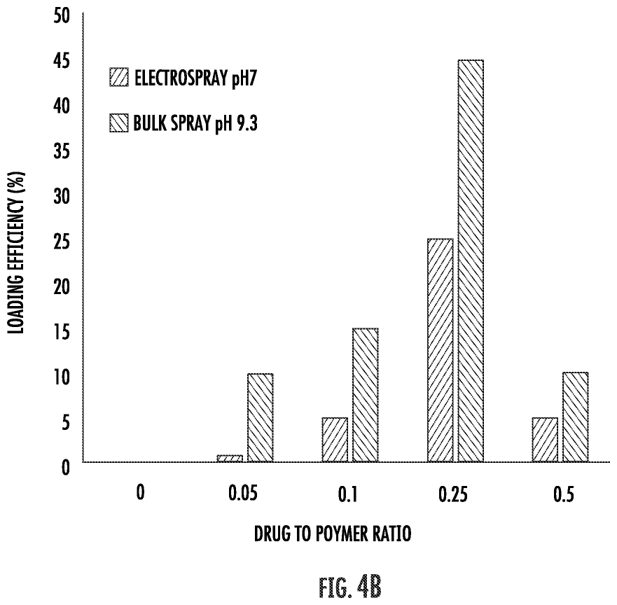

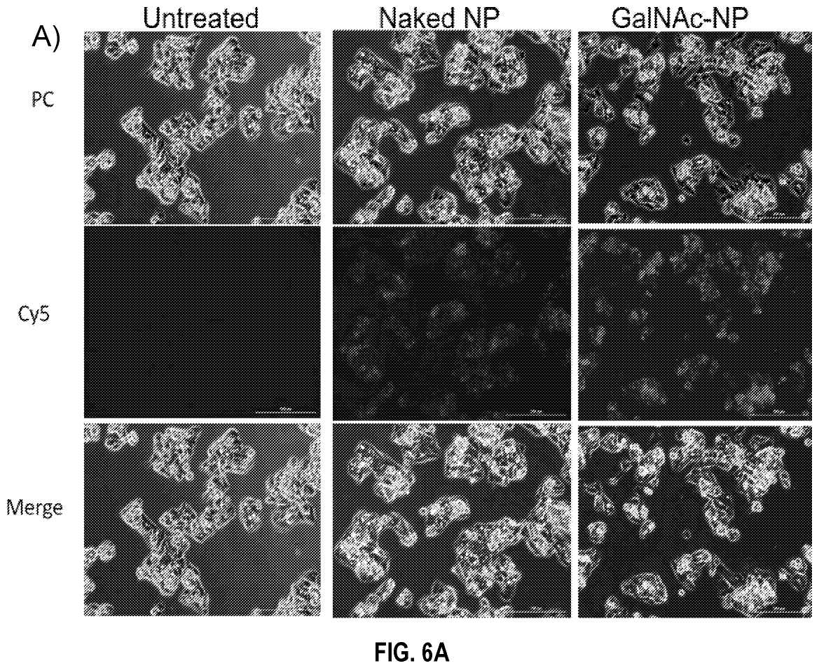

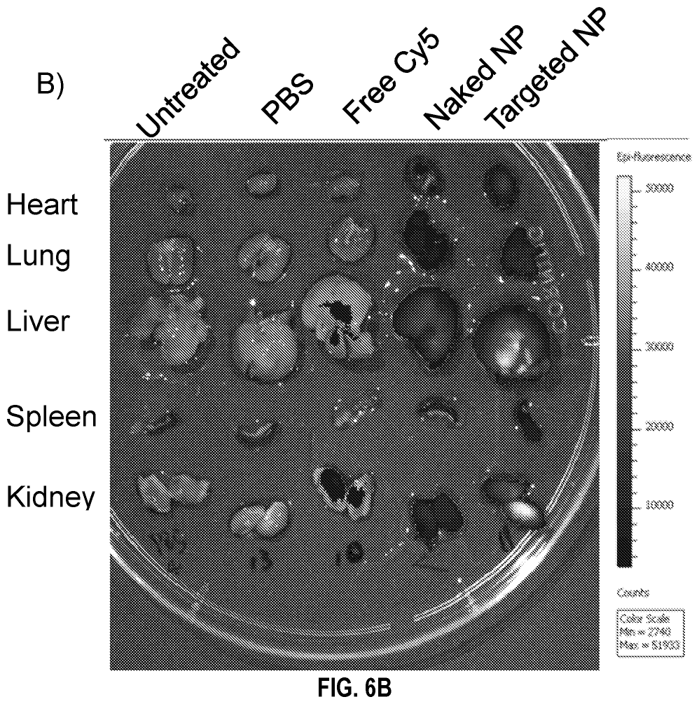

CD38 is widely expressed in hepatocytes as analyzed with imagestream (FIG. 1). Inhibition of CD38 by 78C is protective against hepatic IRI in a short duration 70% partial liver mouse hilar clamp model (FIG. 2A, B, C). A schematic illustration of the precipitation process for GalNAc-PLGA nanoparticles is shown in FIG. 3. PEGylated PLGA NPs are approximately 100 nm in size, with a loading efficiency of 45% by Bulk Spray (FIG. 4A, B). GalNAc-decorated PEGylated PLGA nanoparticles are rapidly taken up by HepG2 cells (FIG. 6A) and demonstrate substantial hepatic uptake on IVIS in mice (FIG. 6B).

### Example 2: Identify and Generate Novel CD38 Inhibitory Compounds

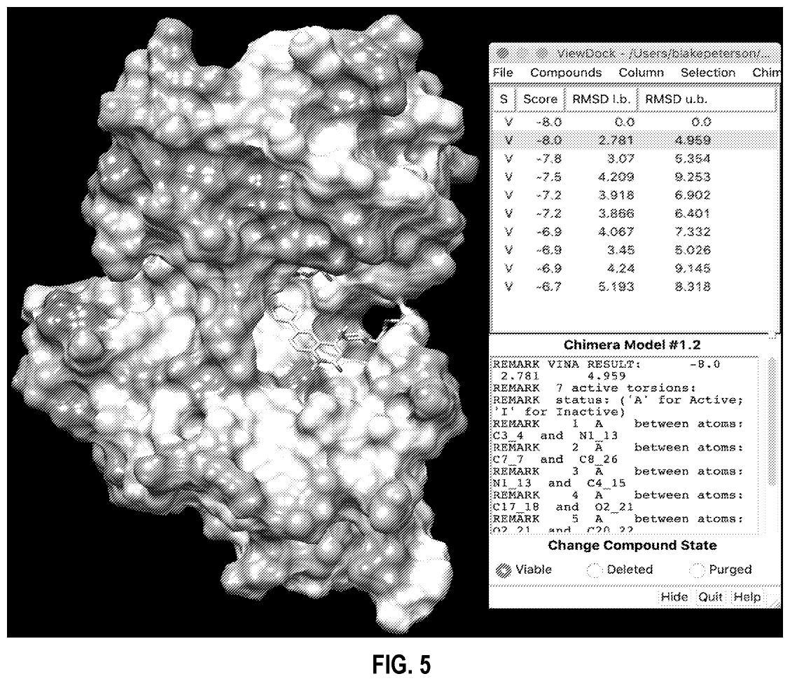

Two different types of fluorescent ligands of CD38 have been reported. The earliest example described fluorophores linked to the highly polar and non-drug-like 2′-deoxy-2′-fluoro arabinosyl NAB (f-ara-NAD). A more recent example described a low-affinity probe comprising an analogue of the more drug-like 78C linked to the fluorophore fluorescein via a long PEG12-CO2H linker. Given the potential of 78C as a therapeutic, we propose to develop novel high-affinity fluorescent probes related to 78C as assays for compound screening and probes of CD38 biology. To improve affinity and create novel fluorescence-based assays for drug discovery, we can use the Autodock vina algorithm and X-ray crystal structures of CD38 to design novel probes and inhibitors. As shown in FIG. 5, we have used this approach to build models of CD38 bound to 78C.

### Example 3: Characterize Protection from IRI by GalNAc Nanoparticles vs. Free CD38 Inhibitor(s) in Relevant Laboratory-Established Small Animal, IRI, and Liver Transplant Models

The CD38 inhibitor 78C, both commercially available and synthesized locally, and hydrophobic analogues generated herein can be encapsulated in biodegradable PLGA nanoparticles that are PEGylated then functionalized with the GalNAc ligand to target hepatocytes (FIG. 5). The conjugation efficiency of GalNAc can then be analyzed by liquid chromatography/mass spectrometry (LC/MS). Optimization GalNAc-CO peptide targeting efficiency can be made by adjusting the ratio of the GalNAc-CO with the NPs. Competitive inhibition experiments can then be performed by adding free GalNAc to a cellular uptake assay to determine uptake efficiency. Pharmacokinetic studies of 78C-encapsulated NPs can then be performed in rats. Rats can be used because multiple blood draws have to be performed for the duration of the experiment. Bolus doses of 78C-NPs can be injected IV. Serum samples can be withdrawn from the ophthalmic vein at various time points over the course of 24 h to measure the plasma concentration of 78C by HPLC. We can also perform in-vitro viability hypoxia-reoxygenation and toxicity (e.g., MTT, cytotoxic green) cell-based assays to determine the effectiveness and toxicity of 78C-NPs compared with vehicle controls and free 78C.

Using the data from the effective dose and toxicity assays, 70% liver hilar clamp IRI experiments (1-hour ischemia with up to 48 hours reperfusion) can be performed in C57BL/6 mice and Lewis Rats, aged 6-8 weeks. The treatment groups can include saline, free 78C, 78C—naked NPs, and 78C—GalNAc NPs. Hepatic tissue can then be collected for H&E, PCR, WB, and other studies (NAD/NADH ratio, MDA, GSH, ATP, ALT, AST, cADPR, NAADP, and CD38 activity assay). PCR and WB can be used to assess RNA and protein expression of downstream targets of CD38. These experiments can provide important information on the effectiveness of selective hepatic CD38 inhibition in a well-established model of warm ischemia. We can then take the findings of the IRI experiments and perform rat liver transplantation using 30 minutes of warm ischemia and 2 hours of cold ischemia prior to transplantation. Animals are maintained for up to 72 hours prior to euthanasia and tissue/specimen collection. The treatment groups can include saline, free 78C, 78C—naked NPs, and 78C—GalNAc NPs. The treatments can be administered either pre-procurement, during cold preservation, prior to reperfusion, and post-transplantation to determine the therapeutic window and overall effectiveness.

A fluorescence-based assay that can quantify the binding of inhibitors to CD38 was developed. This assay is based on the synthesis of a derivative of the CD38 inhibitor 78C linked to the small coumarin-derived fluorophore Pacific Blue (PB). The design of our fluorescent probe PB-78C was facilitated by docking studies using a structure of CD38 (4xjs) and the Autodock viva docking algorithm. Experimentally, we can observe these compounds binding to cell surfaces in an NAD-dependent manner by confocal microscopy and its competition away from cells upon treatment with 78C. Using saturation binding assays by flow cytometry, we showed that different fluorescent probes comprising PB-78C bind CD38 on the surface of Daudi cells with an affinity (Kd) of approximately 0.02-2 micromolar depending on the linker, and these fluorescent probe can be used in competition binding experiments to rapidly identify other non-fluorescent competitive inhibitors by flow cytometry. The Ki value for the published 78C inhibitor measured with our assay (Ki=5.2. nM) is essentially identical to the known Ki value for this compound in enzymatic assays (Ki=7.3 nM, J. Med. Chem. 2015, 58, 3548-3571). This could be used for rapid screening to identify and quantify other inhibitors. We also synthesized a red fluorescent resorufamine derivative of 78C termed RA-78C, and a green-fluorescent Pennsylvania Green derivative of 78C (PG-78C), but non-specific binding made these compounds less effective as probes for CD38 assays. However, these compounds provide additional SAR information by determination of Ki values in the 78C assay. The structures of these compounds and calculated and measured properties are shown in the attached files. The Black lab additionally tested three fluorescent probes in a commercial enzymatic CD38 hydrolase assay, but these compounds proved to inhibit the enzyme at similar low nM levels, in contrast to our cell-based assay, which showed substantial differences in affinity, indicating that the commercial assay is not likely to be sufficiently sensitive to profile novel inhibitors.

Using computational docking and properties-based drug design methods, we further designed a series of novel small molecule CD38 inhibitors that are structurally related to 78C. We are particularly interested in desamino analogues of 78C such as the compounds described. Searching the Scifinder database indicates that no similar desamino compounds have been previously synthesized. We are in the process of synthesizing these compounds and evaluating them in our CD38 binding assay. Compounds with the best properties will be developed as novel immunomodulators that target CD38 and improve outcomes for organ transplantation.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

### Example 4: CD38 Inhibition and Macrophage and Animal Transplant

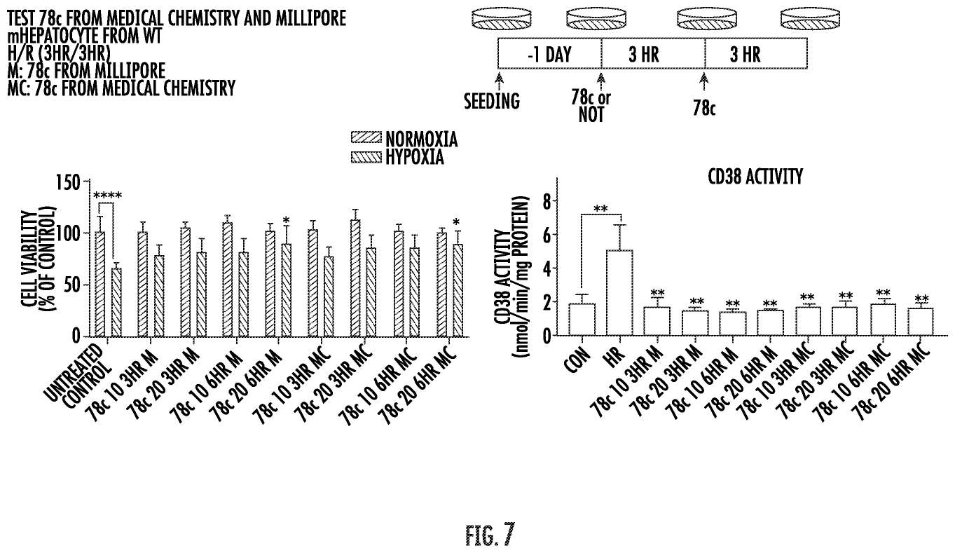

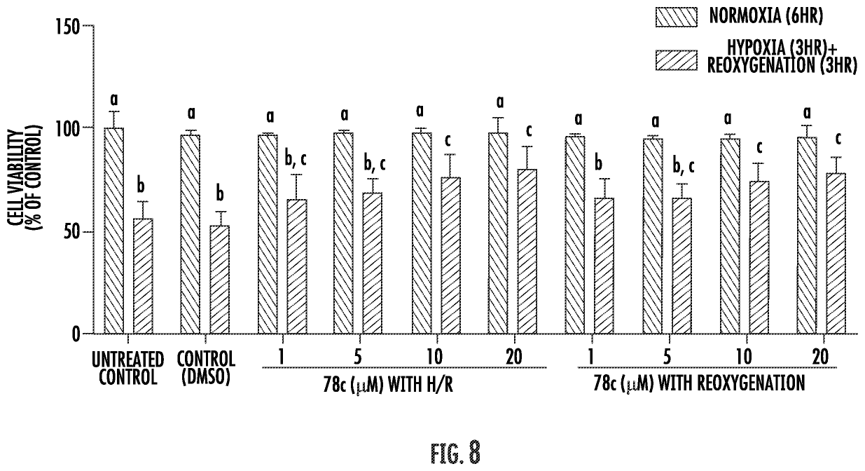

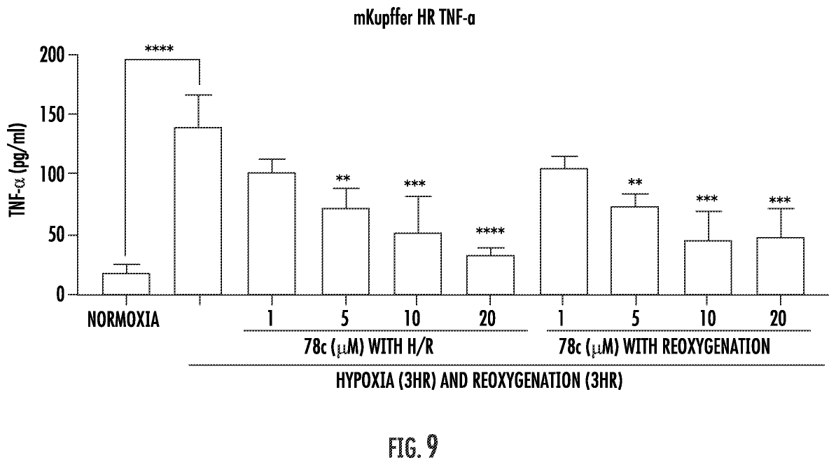

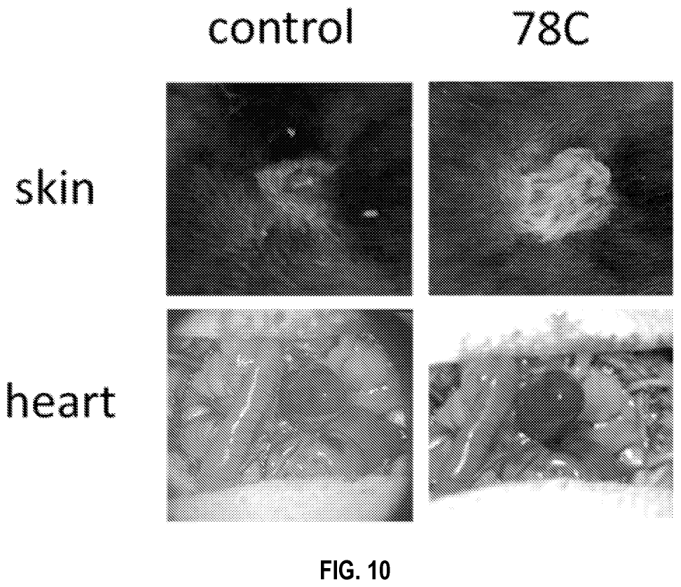

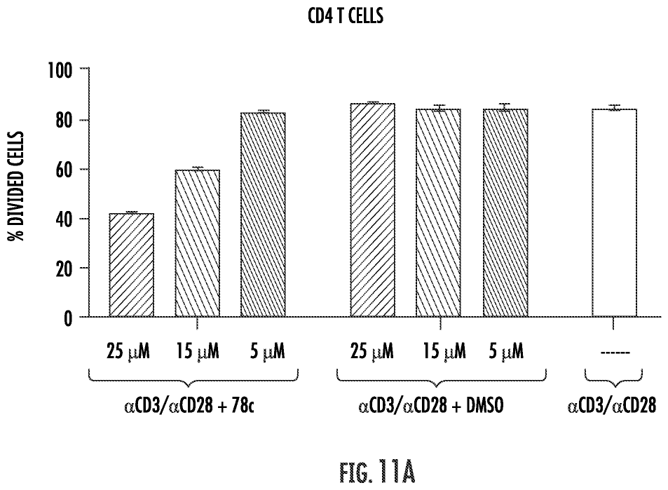

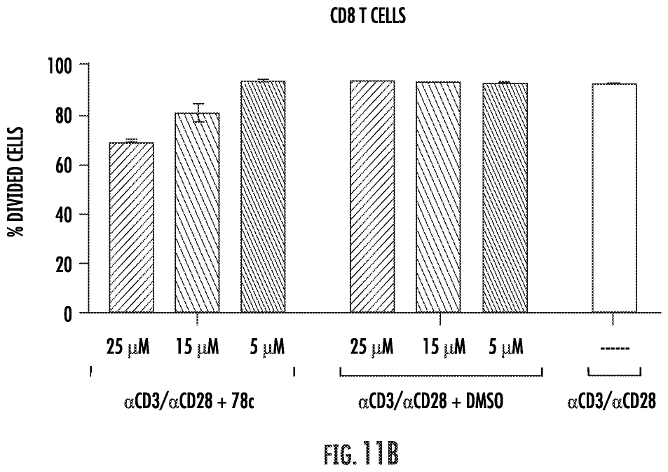

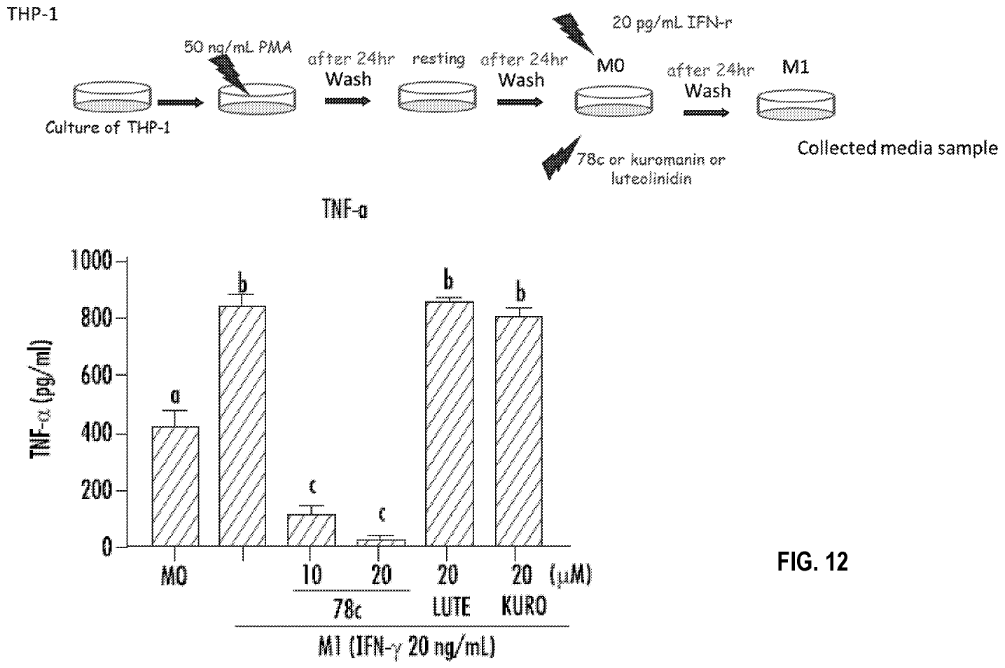

Primary hepatocytes cell viability and CD38 activity following 78c administration was tested (FIG. 7). In untreated cells 3 hours of hypoxia and reoxygenation induced cell death and increased CD38 activity. However, in the 78c treated group CD38 activity decreased and cell viability remained constant. The viability of mouse of mouse kupffer cells (a phagocytic macrophage cell in liver parenchyma) exposed to hypoxia-reoxygenation and treated with increasing dose of 78c was also examined (FIG. 8). In all cases viability increased relative to untreated controls. Additionally, INF-a expression was examined demonstrating blunting of the inflammatory response (FIG. 9). Lastly, to test the ability of 78c to prolong allograft survival, wild-type C57BL/6 mice received fully MHC mismatched A/J skin or heterotopic cardiac allografts. Mice were treated with 10 mg/kg 78c or vehicle delivered IP for 7 (heart) or 14 (skin) days. Representative photographs show skin allografts 17 days post-transplant and heart allografts 7 days post-transplant. 78C treated mice maintained viable skin and cardiac allografts compared with vehicle treated controls (FIG. 10).

### REFERENCES

Quarona, V. et al. CD38 and CD157: A long journey from activation markers to multifunctional molecules. Cytom Part B Clin Cytom 8413, 207 217 (2013).
Aksoy, P. et al. Regulation of SIRT 1 mediated NAD-dependent deacetylation: A novel role for the multifunctional enzyme CD38. Biochem Bioph Res Co 349, 353 359 (2006).
Singha, K., Namgung, R. & Kim, W. J. Polymers in Small-Interfering RNA Delivery. Nucleic Acid Ther 21, 133-147 (2011).
Danhier, F. et al. PLGA-based nanoparticles: An overview of biomedical applications. J Control Release 161, 505-522 (2012),
Han, J.-H., Oh, Y.-K., Kim, D,-S. & Kim, C.-K. Enhanced hepatocyte uptake and liver targeting of methotrexate using galactosylated albumin as a carrier. Int J Pharmaceut 188, 39-47 (1999).
Davis, B. G. & Robinson, M. A. Drug delivery systems based on sugar-macromolecule conjugates. Curr Opin Drug Disc 5, 279-88 (2002).
Scully, S. S. et al. Synthesis and Evaluation of Thiazoloquinolinones with Linkers To Enable Targeting of CD38. Acs Med Chem Lett 8, 196-200 (2016).
Shrimp, J. H. et al. Revealing CD38 Cellular Localization Using a Cell Permeable, Mechanism-Based Fluorescent Small-Molecule Probe. J Am Chem Soc 136, 5656-5663 (2014).
Trott, O. & Olson, A. J. AutoDock Vina: Improving the speed and accuracy of docking with a new scoring function, efficient optimization, and multithreading. J Comput Chem 31, 455-461 (2010).
Becherer, J. D. et al. Discovery of 4-Amino-8-quinoline Carboxamides as Novel, Submicromolar Inhibitors of NAD-Hydrolyzing Enzyme CD38. J Med Chem 58, 7021 7056 (2015).
Hu, Y., Stumpfe, D. & Bajorath, J. Recent Advances in Scaffold Hopping: Miniperspective. J Med Chem 60, 1238-1246 (2016).

What is claimed is:

1. A method of treating, inhibiting, decreasing, reducing, ameliorating and/or preventing a donor organ or tissue rejection in a recipient subject in need thereof, the method comprising administering to the recipient subject an effective amount of a small molecule CD38 inhibitor to improve the organ or tissue transplant outcome.

2. A method of preparing a donor organ or tissue for transplant comprising administering to a recipient subject an effective amount of a small molecule CD38 inhibitor.

3. A method of treating, inhibiting, decreasing, reducing, ameliorating and/or preventing inflammatory injury to a donor organ or tissue in a recipient subject comprising administering to the recipient subject an effective amount of a small molecule CD38 inhibitor.

4. The method of any one of claims 1-3, wherein the recipient subject is resistant or has acquired resistance to immunosuppressive treatment with one or more of eculizumab, thymoglobulin, bortezomib, carfilzomib, basiliximab, mycophenolate mofetil, tacrolimus, or corticosteroids.

5. The method of any one of claims 1-4, wherein the organ or tissue is one or more of heart, liver, lung, pancreas, kidney, skin, trachea, bone marrow, tendons, cornea, vascular tissue, heart valves, intestines, or a combination thereof.

6. The method of any one of claims 1-5, wherein the CD38 inhibitor is administered before or at the time of an organ transplantation.

7. The method of any one of claims 1-6, wherein the CD38 inhibitor is administered after an organ transplantation.

8. A method of treating, inhibiting, decreasing, reducing, ameliorating and/or preventing graft versus host disease (GvHD) in a subject comprising administering to the subject an effective amount of a small molecule CD38 inhibitor.

9. A method of treating, inhibiting, decreasing, reducing, ameliorating and/or preventing ischemia reperfusion injury (IRI) in a subject comprising administering to the subject an effective amount of a small molecule CD38 inhibitor.

10. A method of treating, inhibiting, decreasing, reducing, ameliorating and/or preventing CD activity in a subject comprising administering to the subject an effective amount of a small molecule CD38 inhibitor.

11. A method of treating an inflammatory disease comprising administering to a subject with an inflammatory disease an effective amount of a small molecule CD38 inhibitor.

12. The method of any of claims 1-11, wherein the CD38 inhibitor is administered orally, topically, intravenously, subcutaneously, transcutaneous, transdermally, intramuscularly, intradermally, intraventricularly, intracranially, intraperitoneally, or a combination thereof.

13. A method of inhibiting CD38 activity in a cell expressing CD38 comprising contacting the cell with an effective amount of a small molecule CD38 inhibitor.

14. A method of treating, inhibiting, decreasing, reducing, ameliorating and/or preventing a donor organ or tissue rejection, the method comprising administering an effective amount of a small molecule CD38 inhibitor to the donor organ or tissue ex vivo to improve the organ or tissue transplant outcome.

15. A method of preparing a donor organ or tissue for transplant comprising administering to the donor organ or tissue an effective amount of a small molecule CD38 inhibitor.

16. A method of treating, inhibiting, decreasing, reducing, ameliorating and/or preventing an inflammatory injury to a donor organ or tissue, the method comprising administering to the donor organ or tissue an effective amount of a small molecule CD38 inhibitor.

17. A method of treating, inhibiting, decreasing, reducing, ameliorating and/or preventing ischemia reperfusion injury (IRI) in a donor organ or tissue comprising administering to the donor organ or tissue an effective amount of a small molecule CD38 inhibitor.

18. The method of any one of claims 14-17, wherein the organ or tissue is one or more of heart, liver, lung, pancreas, kidney, skin, trachea, bone marrow, tendons, cornea, vascular tissue, heart valves, intestines, or a combination thereof.

19. The method of any one of claims 14-18, wherein the CD38 inhibitor is administered before or at the time of an organ transplantation.

20. The method of any one of claims 1-19, wherein the small molecule CD38 inhibitor has a molecular weight of from 100 D to 5 kDa, such as from 100 D to 2 kDa, from 100 D to 1.5 kDa, or from 100 D to 1 kDa.

21. A small molecule CD38 inhibitor for treating, inhibiting, decreasing, reducing, ameliorating and/or preventing a donor organ or tissue rejection in a recipient subject in need thereof.

22. A small molecule CD38 inhibitor for treating, inhibiting, decreasing, reducing, ameliorating and/or preventing an inflammatory injury to a donor organ or tissue in a recipient subject,

23. The small molecule CD38 inhibitor of any one of claims 21-22, wherein the recipient subject is resistant or has acquired resistance to immunosuppressive treatment with one or more of eculizumab, thymoglobulin, bortezomib, carfilzomib, basiliximab, mycophenolate mofetil, tacrolimus, or corticosteroids.

24. The small molecule CD38 inhibitor of any one of claims 21-23, wherein the organ or tissue is one or more of heart, liver, lung, pancreas, kidney, skin, trachea, bone marrow, tendons, cornea, vascular tissue, heart valves, intestines, or a combination thereof.

25. A small molecule CD38 inhibitor for treating, inhibiting, decreasing, reducing, ameliorating and/or preventing graft versus host disease (GvHD) in a subject in need thereof.

26. A small molecule CD38 inhibitor for treating, inhibiting, decreasing, reducing, ameliorating and/or preventing of ischemia reperfusion injury (IRI) in a subject in need thereof.

27. A small molecule CD38 inhibitor for treating, inhibiting, decreasing, reducing, ameliorating and/or preventing CD38 activity in a subject in need thereof.

28. A small molecule CD38 inhibitor for treating an inflammatory disease in a subject in need thereof.

29. A small molecule CD38 inhibitor for treating, inhibiting, decreasing, reducing, ameliorating and/or preventing an inflammatory injury to a donor organ or tissue,

30. A small molecule CD38 inhibitor for treating, inhibiting, decreasing, reducing, ameliorating and/or preventing ischemia reperfusion injury (IRI) in a donor organ or tissue. 31 The small molecule CD38 inhibitor of any one of claims 25-30, wherein the organ or tissue is one or more of heart, liver, lung, pancreas, kidney, skin, trachea, bone marrow, tendons, cornea, vascular tissue, heart valves, intestines, or a combination thereof.

32. The small molecule CD38 inhibitor of any one of claims 21-31, wherein the small molecule CD38 inhibitor has a molecular weight of from 100 D to 5 kDa, such as from 100 D to 2 kDa, from 100 D to 1.5 kDa, or from 100 D to 1 kDa.
